(19) United States
(12) Patent Application Publication
(10) Pub. No.: US 2021/0360768 A1
(43) Pub. Date: Nov. 18, 2021

(54) DRIVER CIRCUIT FOR A DIELECTRIC BARRIER DISCHARGE PLASMA TREATMENT

(71) Applicant: PlasmaCure B.V., Nijmegen (NL)

(72) Inventors: Wouter Bastiaan ZEPER, Eindhoven (NL); Paulien SMITS, Eindhoven (NL); Johannes Pieter DE PENNING, Eindhoven (NL); Matthijs Andreas VAN OORT, Tilburg (NL)

(21) Appl. No.: 17/277,678

(22) PCT Filed: Sep. 20, 2019
(86) PCT No.: PCT/NL2019/050629

§ 371 Date: Mar. 18, 2021

(30) Foreign Application Priority Data
Sep. 20, 2018 (NL) 2021675

(51) Int. Cl. H05H 1/24; A61N 1/04; A61L 2/14; A61B 18/04

(52) U.S. Cl. CPC H05H 1/2406; A61N 1/0464; H05H 2245/30; A61B 18/042; H05H 2240/20; A61L 2/14

(57) ABSTRACT

The invention relates to an electrode arrangement to be coupled to a high voltage source for a dielectric barrier discharge plasma treatment of a to be treated tissue of a patient, which treatment surface is used as a counter electrode, having a plasma generating to be coupled to the high voltage source via a first lead; a dielectric shielding the plasma generating from the surface to be treated; a spacer defining a structured surface on a side of said arrangement facing a surface to be treated, said plasma generating being fitted to the object to be treated and brought in contact with the dielectric, a driver circuit for driving the plasma generating coupled to said high voltage source, wherein the driver circuit drives the plasma generating in a first voltage; said driver arranged to simultaneously drive the plasma generating at a second voltage, wherein first and second voltages combined do not exceed a range of 3-8 k V.

### FIELD OF THE INVENTION

This invention relates to devices for generating non-thermal plasma. In particular, the invention relates to devices that can be applied for treatment of living tissue.

### BACKGROUND OF THE INVENTION

Cold plasmas have considerable potential for skin conditioning, disinfection of skin and wound healing. This is due to the reactive species as well as the electric field that are produced. The skin will be temporary exposed to the plasma to kill microorganisms and to stimulate the human skin and immune cells (e.g. improve cell proliferation) and the microcirculation of the blood.

A problem related to the plasma discharge, is that, due to energy absorption in the skin, it is not possible to full-time continue with plasma discharge. Thus, a pulsed discharge is provided. It is an aim to more efficiently use the electric field, without compromising the treated tissue due to over exposure.

### SUMMARY OF THE INVENTION

In summary, embodiments of the invention pertain to an electrode arrangement for a dielectric barrier discharge plasma treatment of a to be treated tissue of a patient, which treatment surface is used as a counter electrode, having
a plasma generating to be coupled to the high voltage source via a first lead;
a dielectric shielding the plasma generating from the surface to be treated;
a spacer defining a structured surface on a side of said arrangement facing a surface to be treated,
said plasma generating being fitted to the object to be treated and brought in contact with the dielectric,
a high voltage driver circuit for driving the plasma generating, wherein the driver circuit drives the plasma generating in first periods wherein a first voltage is applied to the plasma generating; said driver further arranged to drive the plasma generating in second periods wherein a second voltage is applied to the plasma generating, wherein both first and second voltages do not exceed a range of 3-8 kV and wherein the first voltage creates a dielectric barrier discharge plasma to the treatment surface; and the second voltage does not create a dielectric barrier discharge plasma. The first voltage may produce reactive species and electric fields that kill microbes and/or stimulate human cells, to disinfect skin and/or aid wound healing. The second voltage may produce only electric fields that kill microbes and/or stimulate human cells. These effects can be promoted by plasmas and electric fields with different intensity and HV pulse durations. In an embodiment, in an off-period, the plasma and electric fields fully disappear, and the thermal energy can be dissipated. The plasma treatment may thus be given in multiple intervals with an intermediate period without plasma or electric fields to give time to dissipate the heat produced by the plasma in order to prevent heating up of the skin to an uncomfortable or damaging level. The heat is produced only during the time the plasma or electric fields are present, induced by the power. Since there is no plasma or electric fields present during the off-period, the treatment is not fully effective during the treatment time. Some of the reactive species formed by the plasma will still be present during the off-period, but the electric field will be fully absent.

### BRIEF DESCRIPTION OF THE DRAWINGS

FIG. 1 shows a schematic perspective view of a system configuration of the cold plasma device;

FIG. 2 shows a first schematic example of a new voltage control scheme for a high voltage driving circuit;

FIG. 3 shows a first schematic example of a high voltage driving circuit;

FIG. 4 shows an exemplary schematic for the PWM (pulse width modulation) pulse inputs of the high voltage driving circuit; and

FIG. 5 shows an exemplary form of the generated voltages on the load for the PWM pulse inputs of FIG. 4.

### DETAILED DESCRIPTION

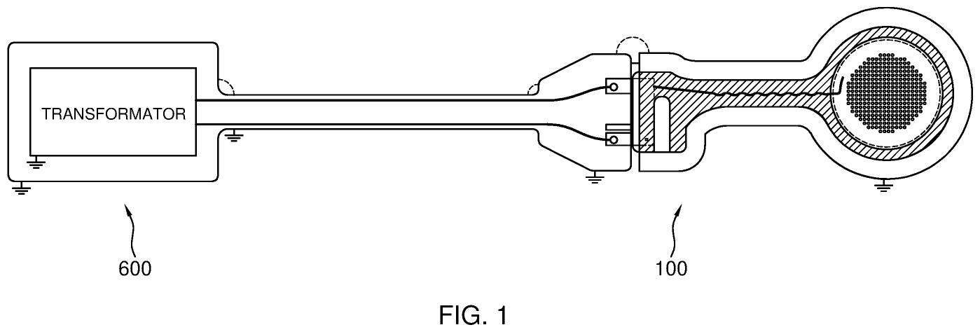

FIG. 1 shows a schematic perspective view of a prototype of the cold plasma device. The plasma device 100 provides a dielectric barrier discharge (DBD) technique for plasma generation. The plasma can be powered by repetitive, short high-voltage (HV) pulses (0.1 ns-10 ms duration, up to a few 100 kHz repetition rate). For example, a driver circuit 600 is provided for driving the planar electrode, wherein the driver circuit drives the planar electrode in a pulsed voltage in a range of 3-8 kV, in a range of 1-100 Hz, and a PWM pulse duration in a range of 50-150 micro seconds. This allows for a pulse rate that substantially provides a micro discharge wherein electrical current through the object to be treated (skin, human body) will only flow during the time that the plasma is on (which is typically equal to the HV pulse duration). In between the HV pulses, the plasma is not active, and no substantial current flows through the skin.

A DBD cold plasma device can treat large areas; the dimensions of the DBD can be chosen over wide margins. Instead of allowing for airflow between the cold plasma device and the skin, discrete compartments may be formed that will contain some air, but these need not be connected to each other. They may be isolated from each other, and may also be isolated to the surroundings by a closed edge.

The advantage of a closed compartment is that the reactive gases that we will generate during operation of the cold plasma, gases like ozone, cannot escape. This has the advantage that the device is more efficient: all reactive specimens are available to kill pathogens and stimulate human cells, and that the release of any toxic gases like ozone will be minimized.

Accordingly an electrode arrangement 100 is shown for a dielectric barrier discharge plasma treatment of an irregularly three-dimensionally shaped surface of an electrically conducting body. The body is typically a human body part, such as a heel, toe, finger or any other diseased skin part, which surface is used as a counter electrode.

The arrangement has a first planar electrode 1 to be coupled to a high voltage source; a dielectric is formed by a flexible material in such a way that the dielectric shields the first planar electrode from the surface to be treated. A spacer structure defines a structured surface on a side of said arrangement 100 facing a surface to be treated.

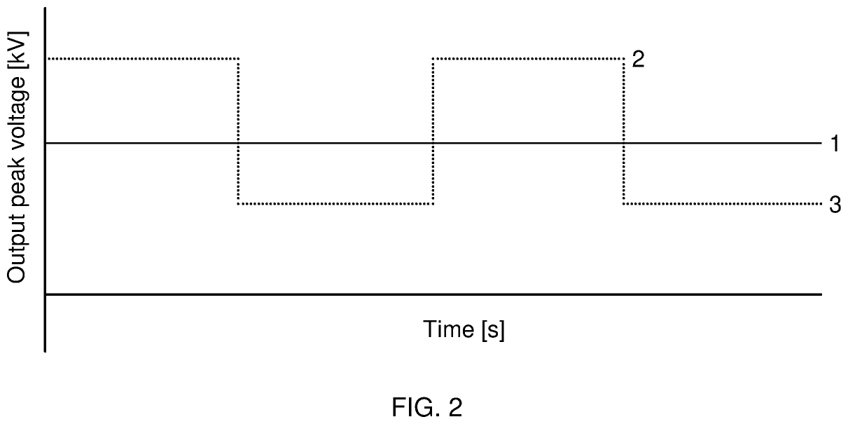

FIG. 2 shows an illustrative output voltage scheme, where it is found that first PWM pulses have a duration of e.g. 50-150 microseconds, with a repetition cycle of e.g. 10 millisecond, e.g. operating on a frequency of 100 Hz, to generate a high voltage (2) above the level needed for DBD discharge (1). Similarly, in the second period, second PWM pulses are generated having a duration of e.g. 5 microsecond-100 microsecond at a repetition rate of 500-1500 Hz. The circuit has characteristics so that this results in a second voltage (3) at a lower level than the first level, typically too little to generate a discharge, but sufficient to generate electric fields up to 10\hat{ }5 V/m.

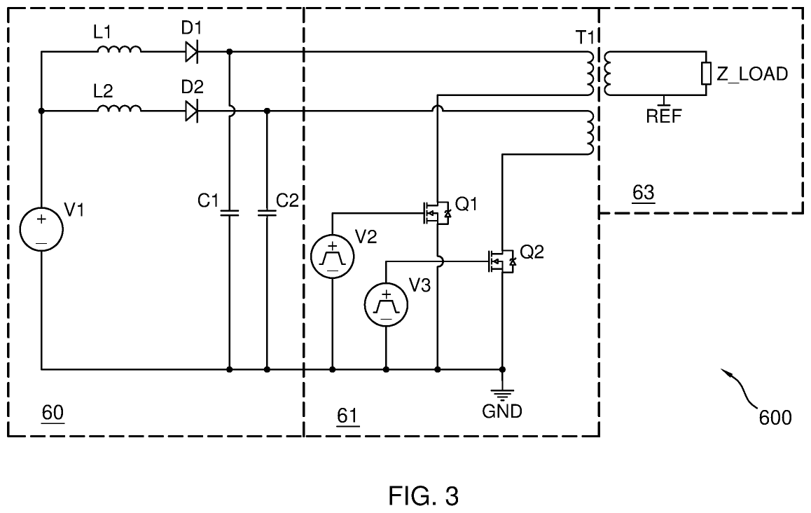

FIG. 3 shows an example driving circuit 600 for driving the plasma generating (ZLoad) in first periods wherein a first voltage is applied to the plasma generating and driving the plasma generating in second periods wherein a second voltage is applied to the plasma generating. The example driving circuit 600 is structured as a pulsed high voltage converter. While various examples can be contemplated and are deemed to be covered by the present disclosure, the circuit generally operates on a power circuit 60 that releases electrical energy into a second control circuit 61, that is accumulated during an active pulse period wherein the electrical energy from a source V1 is inserted in a transformer T1 by a PWM controller V2 and transferred to a third circuit 63 including a secondary wincing of the transformer T1 and the load of the plasma generating. To make it possible to generate HV pulses with different pulse durations an extra control element V3 is added in the driving circuit. The hardware can switch between the two PWM driving components V2 and V3 to get an output with multiple pulse durations. The different HV pulse durations are used, in the first and second periods, to obtain different biological effects. In the first ‘plasma’ period, electric fields above 10\hat{ }5 Vim and HV pulse durations e.g. of 10\hat{ }-10-10\hat{ }-2 seconds can result in either intracellular manipulation, irreversible electroporation or reversible electroporation, depending on the exact electric field and HV pulse duration as well as the size of the biological cell. In the second ‘electric field’ period, electric fields up to 10\hat{ }5 Vim and HV pulse durations e.g. of 10\hat{ }-6-10\hat{ }-2 seconds can result in irreversible or reversible electroporation, again depending on the factors mentioned before. It is noted that these biological effects are in itself known e.g. from Intense picosecond pulsed electric fields induce apoptosis through a mitochondrial-mediated pathway in HeLa cells, Yuan-Yuan Hua et at, DOI 10.3892/mmr.2012.780.

The power circuit 60 includes a power capacitor coupled with the primary winding of the transformer T1. In control circuit 61, a first controllable conductor Q1 is coupled in series to provide a pulsed primary current in the primary winding resonating with the capacitor C1 when the first controllable conductor is switched in a conducting on-state. When the first controllable conductor Q1 is switched in a non conducting off-state the capacitor C1 is fed with electrical current from the voltage source V1.

In the illustrated form, the first power circuit 60 is formed by two power capacitors C1 and C2 in dual circuits each having a diode for unidirectional current flow. The two circuits each generate a different electrical power for driving the second circuit 61 including transformer T1, where the power of the L1C1 circuit is coupled via a first primary wincing, and the power of the L2C2 circuit is coupled via a second primary winding of the transformer T1, resulting in two different waveforms in the third circuit 63, needed for two different HV pulse durations.

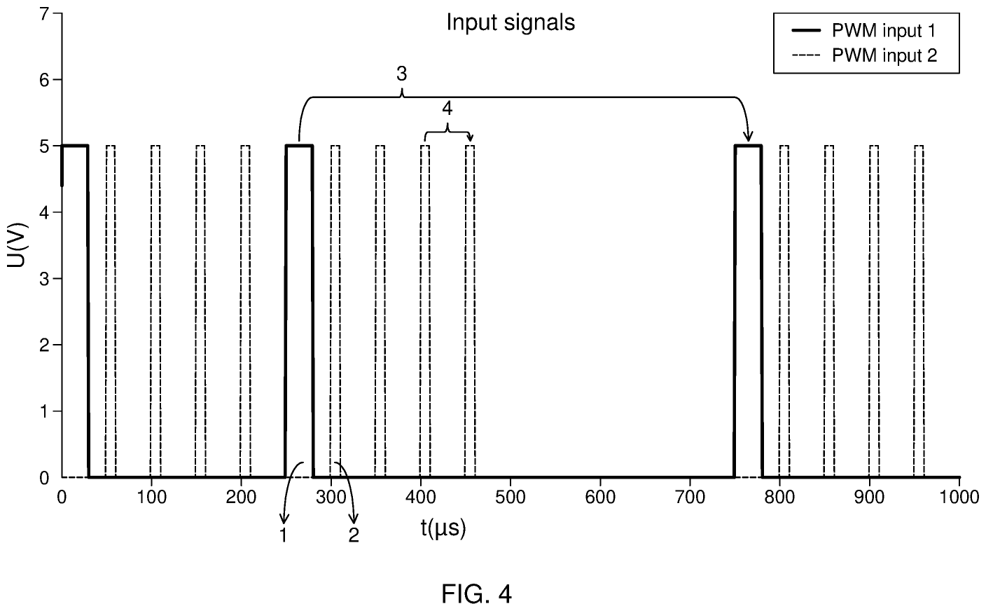

FIG. 4 illustrates an exemplary PWM pulse scheme for driving the high voltage driver circuit of the type of FIG. 3. It is possible to provide a quieter configuration by applying an intermediate field that is provided by second PWM pulses (2) of shorter duration on one of controllable conductors Q1 Q2 of the control circuit 61. The shorter PWM pulses have a repetition rate (4) of about 0.5-1.5 kHz and have a higher repetition rate than first PWM pulses (1) with a longer duration and a lower repetition rate (3) of about 1-100 Hz that is provided by the controller on the other of the controllable conductors Q1, Q2 to control the discharge of the power circuit. As the used voltage for breakdown can be lowered to a lower repetition rate, an accompanying buzzing sound is rendered less audible. To prevent loud and annoying noise during treatment with 1 kHz plasma, the pulse frequency is thus divided into two frequencies: a high frequency, for example 1 kHz and low frequency, for example 100 Hz. The high frequency pulses have a lower voltage so that only an electric field and barely any sound is generated. The low frequency pulses are pulses with plasma and sound, but the sound will be less loud and not annoying due to the lower frequency. Additionally, the electromagnetic radiation produced by the plasma will be reduced.

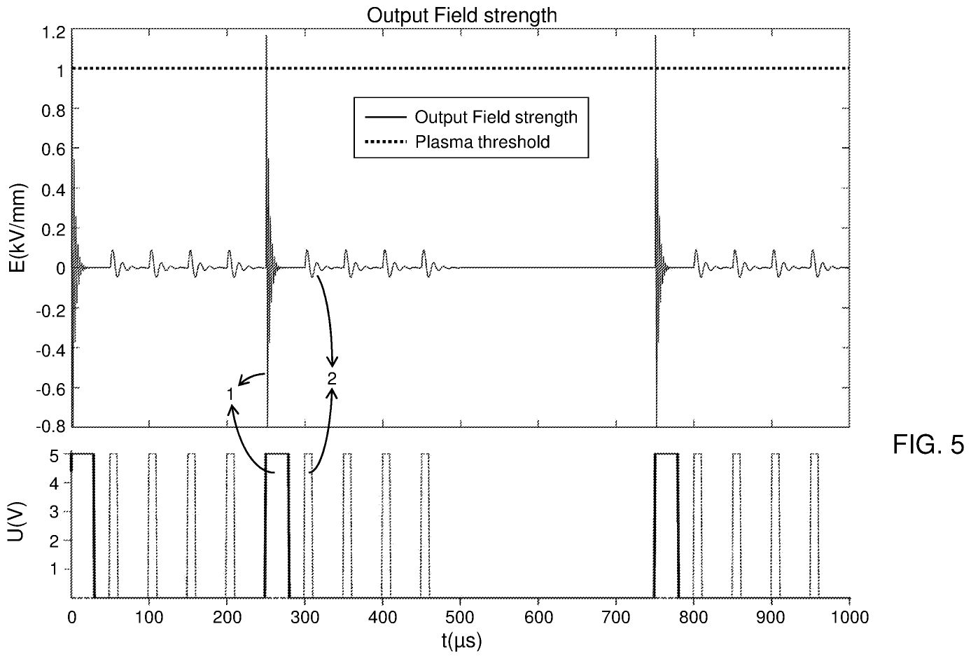

FIG. 5 shows the input plotted simultaneous with a corresponding output in the third circuit, i.e. the voltage measured over the load being the plasma generating electrode. It is shown that the first PWM pulses (1) of longer durations correspond with the plasma generating voltage peaks of higher voltages (e.g. higher than 10\hat{ }5 V/m) where the second PWM pulses (2) of shorter duration correspond to voltage peaks up to 10\hat{ }5 V/m. The relation between voltage output and PWM pulse duration may correspond to circuit specific details and may vary, as long as the PWM pulse duration is long enough to create a high enough voltage to create a plasma.

### Further Embodiments

The method to use the cooling down time effectively is to produce a non-igniting electric field on the electrode by applying a lower voltage than the normal operation voltage on the pad (3). The intermediate period is now used for cooling down as well as for continued stimulation of human cells by applying a continuous electric field.

Intervals and duration of the plasma can be determined by: a fixed program; or based on a measurement, e.g. temperature measurement or reactive species measurement. In this way, a dynamic signal modulation is used to control different operation modes during a treatment. This is used to control the temperature during plasma treatment while maximizing the treatment efficiency and thereby the effectiveness. And using varying frequency to reduce noise. The dual circuits make HV pulse duration variations possible for a single device.

1. An electrode arrangement for a dielectric barrier discharge plasma treatment of a tissue to be treated of a patient, a treatment surface of which is used as a counter electrode, the electrode arrangement having:
a plasma generating electrode to be coupled to a high voltage source via a first lead;
a dielectric that, during operation of the electrode arrangement, shields the plasma generating electrode from the treatment surface of the tissue to be treated; and
a spacer that, during operation of the electrode arrangement, defines a structured surface on a side of said arrangement facing the surface of the tissue to be treated,
wherein said plasma generating electrode is fitted to the patient having the tissue to be treated by operation of the electrode arrangement,
wherein, during operation of the electrode arrangement, the plasma generating electrode is brought in contact with the dielectric,
wherein the electrode arrangement further comprises a high voltage (HV) driver circuit for driving the plasma generating coupled to said plasma generating electrode,
wherein the driver circuit drives the plasma generating in first periods wherein a first voltage is applied to the plasma generating electrode,
wherein the driver circuit is further arranged to drive the plasma generating in second periods wherein a second voltage is applied to the plasma generating,
wherein both the first voltage and the second voltage do not exceed a range of 3-8 kV,
wherein the first voltage in the first periods creates a dielectric barrier discharge plasma; and
wherein the second voltage in the second periods does not create a dielectric barrier discharge plasma.

2. The electrode arrangement according to claim 1, wherein the driver circuit is arranged to provide in respective ones of the first periods and the second periods, a first HV pulse having a first duration differing from a second duration of the second HV pulse.

3. The electrode arrangement according to claim 2, wherein the driver circuit is arranged to provide in the first periods and the second periods a HV pulse duration in a range of 0.1 nano second 10 milli seconds.

4. The electrode arrangement according to claim 1, wherein the driver circuit is equipped with pulse width modulated sources arranged to provide the second voltage at a second repetition rate and/or a second PWM pulse duration that differs from a first repetition rate and/or a first PWM pulse duration of the first voltage.

5. The electrode arrangement according to claim 4, wherein the driver circuit is arranged to provide the first voltage with:
a first repetition rate in a range of 1-100 Hz, and
a first PWM pulse duration in a range of 50-150 micro seconds.

6. The electrode arrangement according to claim 5, wherein the driver circuit is arranged to provide the second voltage in the second periods at a second pulsed frequency, and
wherein the second periods of the second voltage do not overlap the first periods of the first voltage.

7. The electrode arrangement according to claim 6, wherein the driver circuit is configured to pulse the second voltage at:
a frequency in a frequency range of 0.5-1.5 kHz, and
a PWM pulse duration in a range of 5-100 micro seconds.

8. The electrode arrangement according to claim 1, wherein the driver circuit is arranged to provide an off-period where neither the first voltage nor the second voltage is supplied, and
wherein the off-period is alternating with the first period or the second period.
